(19) United States
(12) Patent Application Publication
(10) Pub. No.: US 2021/0058572 A1
(43) Pub. Date: Feb. 25, 2021

(54) IMAGING APPARATUS

(71) Applicant: CANON KABUSHIKI KAISHA, Tokyo (JP)

(72) Inventors: Junya Fukumoto, Yokohama-shi (JP); Yuma Kudo, Kawasaki-shi (JP)

(21) Appl. No.: 16/988,898

(22) Filed: Aug. 10, 2020

(30) Foreign Application Priority Data
Aug. 22, 2019 (JP) 2019-151778

(51) Int. Cl. H04N 5/347; H04N 9/04; H04N 5/353

(52) U.S. Cl. CPC H04N 5/347; A61B 1/042; H04N 5/3532; H04N 9/0451

(57) ABSTRACT

An imaging apparatus includes an optical element configured to separate incident light into light components in at least two types of wavelength bands, and a plurality of imaging elements configured to receive the light components in the at least two types of wavelength bands separated by the optical element, respectively. When pixels that have undergone binning processing are regarded as one pixel on a group basis, pixels of at least one imaging element of the plurality of imaging elements are arranged with a shift of a half pixel in at least one direction of a horizontal direction and/or a vertical direction with respect to pixels of at least one other imaging element.

### BACKGROUND OF THE INVENTION

### Field of the Invention

The present disclosure relates to an imaging apparatus.

### Description of the Related Art

An imaging apparatus, for example, an endoscopic apparatus has conventionally mainly used a CCD (Charge Coupled Apparatus) image sensor. Recently, however, a CMOS (Complementary Metal Oxide Semiconductor) image senor is mainly used because of its advantages such as low cost, single power supply, and low power consumption. As the CMOS image sensor, a rolling shutter method is often employed in general (see Japanese Patent Laid-Open No. 2018-175871).

### SUMMARY OF THE INVENTION

One of problems to be solved by an embodiment disclosed in this specification is to ensure image quality sufficiently for observation. However, the problem is not limited to this, and obtaining functions and effects derived by components shown in a form configured to implement the present invention to be described later can also be defined as another problem to be solved by the embodiment disclosed in this specification and the like.

An imaging apparatus according to the embodiment is an imaging apparatus comprising: an optical element configured to separate incident light into light components in at least two types of wavelength bands; and a plurality of imaging elements configured to receive the light components in the at least two types of wavelength bands separated by the optical element, respectively, wherein when pixels that have undergone binning processing are regarded as one pixel on a group basis, pixels of at least one imaging element of the plurality of imaging elements are arranged with a shift of a half pixel in at least one direction of a horizontal direction and/or a vertical direction with respect to pixels of at least one other imaging element.

Further features of the present invention will become apparent from the following description of exemplary embodiments (with reference to the attached drawings).

### BRIEF DESCRIPTION OF THE DRAWINGS

FIG. 1 is a block diagram showing an example of the configuration of an imaging system including an imaging apparatus according to the embodiment;

FIG. 2 is a view showing an imaging operation in a normal mode as an example of the imaging operation of the imaging apparatus according to the embodiment;

FIG. 3 is a view showing an example of the arrangement of the pixels of the image sensors of the imaging apparatus according to the embodiment;

FIG. 4 is a view showing an example of the arrangement of the pixels of the image sensors when the imaging apparatus according to the embodiment executes an ICG color mode;

FIG. 5 is a view showing an imaging operation in the ICG color mode as an example of the imaging operation of the imaging apparatus according to the embodiment; and

FIG. 6 is a view for explaining a part of the imaging operation in the ICG color mode.

### DESCRIPTION OF THE EMBODIMENTS

An imaging apparatus according to an embodiment will now be described with reference to the accompanying drawings. Note that the embodiment is not limited to the following contents. In addition, the contents described in one embodiment or modification are similarly applied to another embodiment or modification in principle.

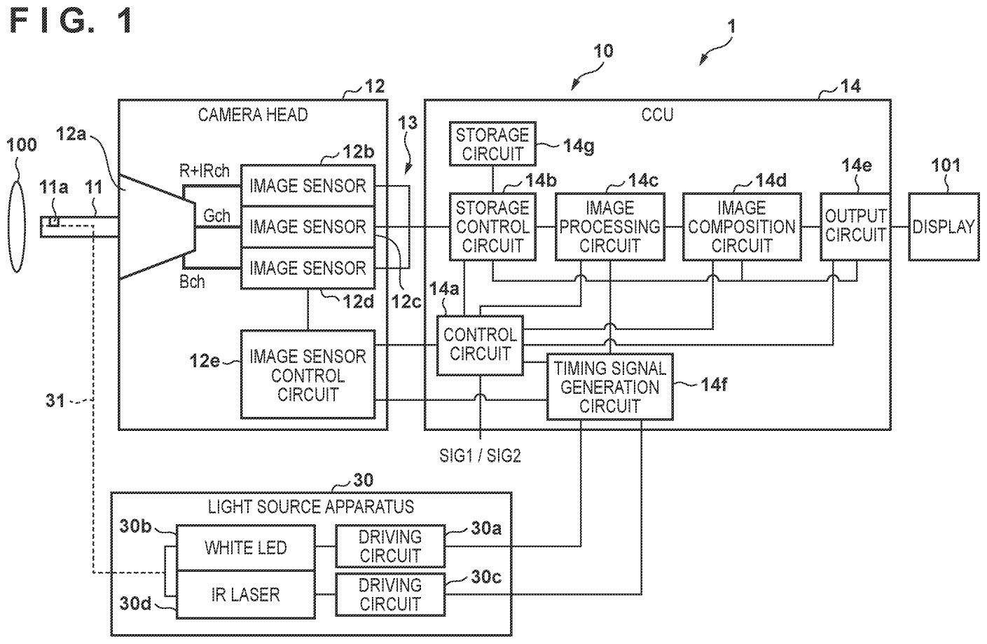

FIG. 1 is a block diagram showing an example of the configuration of an imaging system 1 including an imaging apparatus 10 according to this embodiment. As shown in FIG. 1, the imaging system 1 according to this embodiment includes the imaging apparatus 10, a light source apparatus 30, and an optical fiber 31.

The imaging apparatus 10 is used as, for example, a rigid endoscope for a medical application, which is an apparatus that captures the inside of a subject 100. The imaging apparatus 10 includes a scope 11, a camera head 12, a camera cable 13, and a CCU (Camera Control Unit) 14. Note that the imaging apparatus 10 is not limited only to the rigid endoscope.

The scope 11 is inserted into the inside of the subject 100 when performing imaging. An objective lens 11a is provided at the distal end of the scope 11.

The camera head 12 includes a prism 12a, a plurality of image sensors 12b, 12c, and 12d, and an image sensor control circuit 12e.

The prism 12a separates incident light into light components in two or more types of wavelength bands. For example, the prism 12a is a tricolor separating dichroic prism. For example, the prism 12a spectrally divides incident light into red (R+IR) light, green (G) light, and blue (B) light. The prism 12a is an example of an optical element.

The plurality of image sensors receive the light components in the two or more types of wavelength bands separated by the prism 12a, respectively. For example, the plurality of image sensors are CMOS (Complementary Metal Oxide Semiconductor) image sensors. For example, as the plurality of image sensors, the image sensors 12b, 12c, and 12d receive the red (R+IR) light, the green (G) light, and the blue (B) light separated by the prism 12a, respectively. The image sensor 12b corresponds to, for example, red and infrared wavelength bands (expressed as “R+ IRch (channel)” in FIG. 1), and is provided on the exit surface of the prism 12a for spectrally divided red light. The image sensor 12c corresponds to, for example, a green wavelength band (expressed as “Gch” in FIG. 1), and is provided on the exit surface of the prism 12a for spectrally divided green light. The image sensor 12d corresponds to, for example, a blue wavelength band (expressed as “Bch” in FIG. 1), and is provided on the exit surface of the prism 12a for spectrally divided blue light. The image sensors 12b, 12c, and 12d will sometime be referred to as the image sensor 12b on the R+IRch side, the image sensor 12b on the Gch side, and the image sensor 12b on the Bch side, respectively, hereinafter. The imaging surfaces of the image sensors 12b, 12c, and 12d are arranged to almost match the imaging surface of an optical system including the scope 11. The image sensors 12b, 12c, and 12d are examples of an imaging element.

Each of the image sensors 12b, 12c, and 12d includes a plurality of pixels (imaging pixels). The plurality of pixels are arranged in a matrix on the imaging surface. Under the driving control of the image sensor control circuit 12e, each pixel generates a video signal (electrical signal) by receiving light, and outputs the generated video signal. For example, each pixel of the image sensor 12b receives red light, thereby outputting an R signal (R video signal). In addition, each pixel of the image sensor 12c receives green light, thereby outputting a G signal (G video signal). Furthermore, each pixel of the image sensor 12d receives blue light, thereby outputting a B signal (b video signal). For example, the camera head 12 including the image sensors 12b, 12c, and 12d outputs an RGB signal to the CCU 14 via the camera cable 13. Note that an analog video signal is output from each of the image sensors 12b, 12c, and 12d. Alternatively, if each of the image sensors 12b, 12c, and 12d incorporates an A/D (Analog to Digital) converter (not shown), a digital video signal is output from each of the image sensors 12b, 12c, and 12d.

Here, the imaging apparatus 10 according to this embodiment is used when, for example, performing a surgical operation by ICG (IndoCyanine Green) fluorescence angiography for the subject 100. In this case, ICG is administered to the subject 100. ICG is excited by excitation light emitted by an IR laser 30d and emits near-infrared fluorescence (to be referred to as fluorescence hereinafter) of about 800 to 850 nm. In the ICG fluorescence angiography, a filter that cuts excitation light is provided between the scope 11 and the prism 12a, and the fluorescence is received by the image sensor 12b. That is, the image sensor 12b receives the fluorescence based on the excitation light, thereby outputting an R signal.

Each of the image sensors 12b, 12c, and 12d is a rolling shutter image sensor that repeats, for every frame (image), processing of sequentially starting exposure, at least on each row, from the first row to the final row of the plurality of pixels and outputting a video signal sequentially from a row that has undergone the exposure. Here, exposure means, for example, accumulating charges in the pixels.

The image sensor control circuit 12e drives and controls the image sensors 12b, 12c, and 12d based on a control signal output from a control circuit 14a to be described later and various kinds of synchronization signals output from a timing signal generation circuit 14f to be described later. For example, if the image sensors 12b, 12c, and 12d output analog video signals, the image sensor control circuit 12e appropriately applies a gain (analog gain) to each of the analog video signals output from the image sensors 12b, 12c, and 12d (amplifies the video signals) based on the control signal and the various kinds of synchronization signals, thereby controlling the image sensors 12b, 12c, and 12d such that the video signals multiplied by the gain are output to the CCU 14. Alternatively, if the image sensors 12b, 12c, and 12d output digital video signals, the image sensor control circuit 12e appropriately applies a gain (digital gain) to each of the digital video signals output from the image sensors 12b, 12c, and 12d based on the control signal and the various kinds of synchronization signals, thereby controlling the image sensors 12b, 12c, and 12d such that the video signals multiplied by the gain are output to the CCU 14.

The camera cable 13 is a cable that stores signal lines configured to transmit/receive video signals, control signals, and synchronization signals between the camera head 12 and the CCU 14.

The CCU 14 performs various kinds of image processing for a video signal output from the camera head 12 to generate image data to be displayed on a display 101, and outputs the image data to the display 101 connected to the CCU 14. Note that the video signal that has undergone the various kinds of image processing is image data representing an image to be displayed on the display 101.

The CCU 14 includes the control circuit 14a, a storage control circuit 14b, an image processing circuit 14c, an image composition circuit 14d, an output circuit 14e, the timing signal generation circuit 14f, and a storage circuit 14g. Note that when the image sensors 12b, 12c, and 12d output analog video signals, the CCU 14 includes an A/D converter and the like (not shown) as well. The A/D converter converts, for example, analog video signals output from the image sensors 12b, 12c, and 12d into digital video signals.

The control circuit 14a controls various kinds of constituent elements of the imaging apparatus 10. For example, the control circuit 14a outputs control signals to the image sensor control circuit 12e, the storage control circuit 14b, the image processing circuit 14c, the image composition circuit 14d, the output circuit 14e, and the timing signal generation circuit 14f, thereby controlling the circuits. The control circuit 14a loads the control program of the imaging apparatus 10, which is stored in the storage circuit 14g, and executes the loaded control program, thereby executing control processing of controlling the various kinds of constituent elements of the imaging apparatus 10. Alternatively, the control circuit 14a incorporates a storage circuit (not shown) and executes a control program stored in the storage circuit. The control circuit 14a is implemented by, for example, a processor such as an MPU (Micro-Processing Unit). Here, the control circuit 14a is an example of a switching control unit. In addition, the control circuit 14a and the above-described image sensor control circuit 12e are examples of a driving control unit.

The storage control circuit 14b performs control of storing, in the storage circuit 14g, a video signal output from the camera head 12 based on a control signal output from the control circuit 14a and various kinds of synchronization signals output from the timing signal generation circuit 14f In addition, the storage control circuit 14b reads the video signal stored in the storage circuit 14g from each row based on the control signal and the synchronization signals. The storage control circuit 14b then outputs the read video signal of one row to the image processing circuit 14c.

The image processing circuit 14c performs various kinds of image processing for the video signal output from the storage control circuit 14b based on a control signal output from the control circuit 14a and various kinds of synchronization signals output from the timing signal generation circuit 14f. The image processing circuit 14c thus generates image data representing an image to be displayed on the display 101. That is, the image processing circuit 14c generates the image based on the video signal. For example, the image processing circuit 14c applies a gain (digital gain) to the video signal output from the storage control circuit 14b, thereby adjusting the brightness of the image. The image processing circuit 14c may perform noise reduction processing of reducing noise or edge enhancement processing of enhancing edges for the video signal output from the storage control circuit 14b. The image processing circuit 14c outputs the video signal (image data representing the image to be displayed on the display 101) that has undergone the various kinds of image processing to the image composition circuit 14d. The image processing circuit 14c is an example of a processing unit.

The image composition circuit 14d composites video signals output from the image processing circuit 14c to generate composite image data based on a control signal output from the control circuit 14a and various kinds of synchronization signals output from the timing signal generation circuit 14f. The image composition circuit 14d outputs the composite image data to the display 101.

For example, the storage control circuit 14b, the image processing circuit 14c, and the image composition circuit 14d are implemented by one processor such as a DSP (Digital Signal Processor). Alternatively, for example, the storage control circuit 14b, the image processing circuit 14c, the image composition circuit 14d, and the timing signal generation circuit 14f are implemented by one FPGA (Field Programmable Gate Array). Note that the control circuit 14a, the storage control circuit 14b, the image processing circuit 14c, and the image composition circuit 14d may be implemented by one processing circuit. The processing circuit is implemented by, for example, a processor.

The output circuit 14e outputs the composite image data output from the image composition circuit 14d to the display 101. The display 101 thus displays a composite image represented by the composite image date. The composite image is an example of an image. The output circuit 14e is implemented by, for example, an HDMI® (High-Definition Multimedia Interface) driver IC (Integrated Circuit), an SDI (Serial Digital Interface) driver IC, or the like.

The timing signal generation circuit 14f unitarily manages various kinds of timings such as the emission timing of light from the light source apparatus 30, the exposure timings and video signal output timings of the image sensors 12b, 12c, and 12d, and the control timing of the storage circuit 14g by the storage control circuit 14b.

The timing signal generation circuit 14f generates various kinds of synchronization signals such as a horizontal synchronization signal and a vertical synchronization signal, and other synchronization signals used to synchronize the entire imaging apparatus 10 based on a clock signal generated by an oscillation circuit (not shown). The timing signal generation circuit 14f outputs the generated various kinds of synchronization signals to the image sensor control circuit 12e, the control circuit 14a, the storage control circuit 14b, the image processing circuit 14c, the image composition circuit 14d, and the output circuit 14e.

In addition, the timing signal generation circuit 14f generates a light source control signal based on the clock signal and a control signal output from the control circuit 14a. The light source control signal is a control signal used to control light emitted from the light source apparatus 30 and also synchronize the entire imaging system 1. The timing signal generation circuit 14f outputs the generated light source control signal to the light source apparatus 30.

For example, the light source control signal has a rectangular waveform, and takes two levels (states), that is, high level and low level. For example, the light source control signal is a control signal that causes the light source apparatus 30 to emit light during high level, and stops emission of light from the light source apparatus 30 during low level.

The storage circuit 14g is implemented by, for example, a RAM (Random Access Memory), a ROM (Read Only Memory), a semiconductor memory element such as a flash memory, a hard disk, an optical disk, or the like. The ROM (or flash memory or hard disk) stores various kinds of programs. For example, the ROM stores a control program to be executed by the control circuit 14a. In addition, video signals are temporarily stored in the RAM by the storage control circuit 14b.

The light source apparatus 30 emits white light or excitation light based on the light source control signal. The light source apparatus 30 includes a driving circuit 30a, a white LED (Light Emitting Diode) 30b, a driving circuit 30c, and an IR laser 30d.

The driving circuit 30a performs driving control of driving and turning on the white LED 30b based on the light source control signal output from the timing signal generation circuit 14f. The white LED 30b emits white light under the driving control of the driving circuit 30a. The white light is, for example, visible light.

The driving circuit 30c performs driving control of driving the IR laser 30d and causing the IR laser 30d to emit excitation light based on the light source control signal output from the timing signal generation circuit 14f. The IR laser 30d emits excitation light under the driving control of the driving circuit 30c. Note that fluorescence (fluorescence based on the excitation light) emitted from the ICG excited by the excitation light is received by the image sensor 12b.

The optical fiber 31 guides the white light and the excitation light from the light source apparatus 30 to the distal end portion of the scope 11 and outputs the light from the distal end portion of the scope 11.

An example of the configuration of the imaging apparatus 10 of the imaging system 1 according to this embodiment has been described above. Here, as shown in FIG. 1, the control circuit 14a of the CCU 14 switches between an ICG color mode and a normal mode in accordance with an input switching signal. For example, when performing a surgical operation by administering ICG to the subject 100, a switching signal SIG1 is input to the control circuit 14a. In this case, the control circuit 14a switches the mode to be executed to the ICG color mode in accordance with the switching signal SIG1. For example, when performing a surgical operation without administering ICG to the subject 100, a switching signal SIG2 is input to the control circuit 14a. In this case, the control circuit 14a switches the mode to be executed to the normal mode in accordance with the switching signal SIG2. The ICG color mode is an example of a first mode, and the normal mode is an example of a second mode.

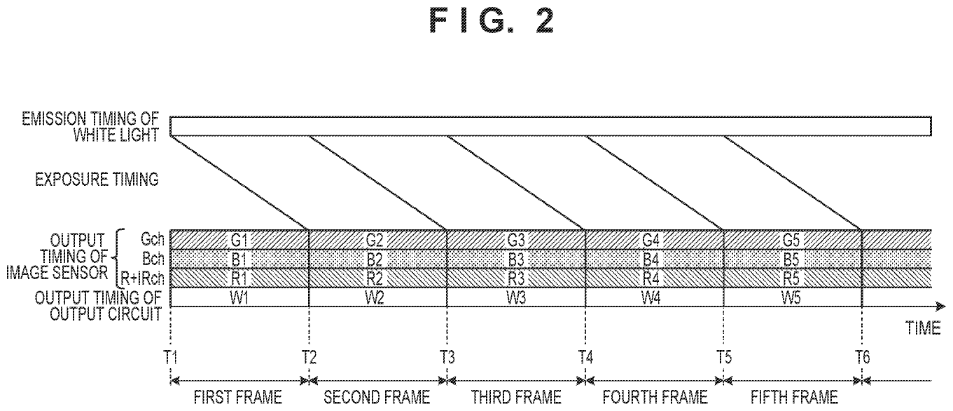

The normal mode will be described first. FIG. 2 is a view showing an imaging operation in the normal mode as an example of the imaging operation of the imaging apparatus 10 according to this embodiment. FIG. 2 shows an example of the relationship between the emission timing of white light emitted from the light source apparatus 30, the exposure timings of the rows of the plurality of pixels provided in the image sensors 12b, 12c, and 12d of the imaging apparatus 10, the output timings of video signals output from the image sensors 12b, 12c, and 12d, and the output timings of a video signal output from the output circuit 14e. In FIG. 2, the abscissa represents time. In the normal mode, the frame rate of a video signal (image) output from the imaging apparatus 10 to the display 101 is 60 [fps (frame per second)], and the read period is 1/60 [s]. That is, the period of outputting a video signal of one frame from the imaging apparatus 10 to the display 101 and the read period are 1/60 [s].

First, at the start of imaging, the control circuit 14a outputs a control signal to the timing signal generation circuit 14f to cause it to output a light source control signal that causes the white LED 30b to continuously emit white light. The timing signal generation circuit 14f outputs the light source control signal to the driving circuit 30a based on the control signal, and the driving circuit 30a drives the white LED 30b based on the light source control signal, thereby causing the white LED 30b to continuously emit white light.

For example, in the first frame, during the read period of 1/60 [s] from time T1 to time T2, exposure is sequentially started on each row from the first row to the final row of the plurality of pixels of each of the image sensors 12b, 12c, and 12d. More specifically, the control circuit 14a outputs a control signal to the image sensor control circuit 12e to cause each of the image sensors 12b, 12c, and 12d to output a video signal during the read period of 1/60 [s]. The image sensor control circuit 12e drives and controls the image sensors 12b, 12c, and 12d based on the control signal. As a result, during the read period of 1/60 [s], the image sensor 12b receives light in the red wavelength band, which has exited from the prism 12a, and outputs video signals from all rows as an R signal “R1”. The image sensor 12c receives light in green wavelength band, which has exited from the prism 12a, and outputs video signals from all rows as a G signal “G1”. The image sensor 12d receives light in blue wavelength band, which has exited from the prism 12a, and outputs video signals from all rows as a B signal “B1”. In this case, an RGB signal “W1” is output as a video signal from each of the image sensors 12b, 12c, and 12d. The RGB signal “W1” represents the composite signal of the R signal “R1”, the G signal “G1”, and the B signal “B1”. That is, the RGB signal “W1” includes the R signal “R1” output from the image sensor 12b that has received the white light, the G signal “G1” output from the image sensor 12c that has received the white light, and the B signal “B1” output from the image sensor 12d that has received the white light.

Next, in the second frame, during the read period of 1/60 [s] from time T2 to time T3, the image sensors 12b, 12c, and 12d output video signals as an R signal “R2”, a G signal “G2”, and a B signal “B2”, respectively. In this case, an RGB signal “W2” is output as a video signal from each of the image sensors 12b, 12c, and 12d. The RGB signal “W2” represents the composite signal of the R signal “R2”, the G signal “G2”, and the B signal “B2”.

Here, the video signals output from the image sensors 12b, 12c, and 12d are changed to the display image of the first frame via the image processing circuit 14c and the image composition circuit 14d, and quickly output from the output circuit 14e to the display 101. More specifically, the image processing circuit 14c generates a display image based on the RGB signal “W1” of the first frame. The display image generated by the image processing circuit 14c is output from the output circuit 14e to the display 101 during the period of 1/60 [s]. From the second frame as well, processing similar to the above-described processing is performed.

On the other hand, in the ICG color mode, in each frame, time-divisional control is performed in which an R signal “IR” is acquired during the first read period, and an RGB signal is acquired during the second read period. In the ICG color mode, since time-divisional control is performed, the image sensors 12b, 12c, and 12d need to be driven and controlled at a speed higher than in the normal mode. For example, in the ICG color mode, when the frame rate of a video signal (image) output from the imaging apparatus 10 to the display 101 is 60 [fps], the read period is set to 1/120 [s].

Here, a technique called binning processing is known. In binning processing, adjacent pixels are added, thereby decreasing the number of pixels to be read. For example, in the ICG color mode, the image sensors 12b, 12c, and 12d can easily be driven at a high speed by performing binning processing.

However, it is known that when binning processing is performed, the resolution of an image lowers in general. That is, in the ICG color mode, when binning processing is performed, the image sensors 12b, 12c, and 12d can easily be driven at a high speed, but the resolution lowers. Hence, for a user such as a doctor who observes images, image quality may not be sufficient for observation.

Also, in the ICG color mode, if pixels as many as in the normal mode are read by time-divisional control, the number of signal processing operations becomes large. Hence, if the diameter per cable such as the camera cable 13 or the number of cables is increased because of the increase in the number of signal processing, the diameter of the entire cable becomes large.

The imaging apparatus 10 according to this embodiment performs the following processing to ensure image quality sufficient for the user to observe. The imaging apparatus 10 according to this embodiment includes the prism 12a, and the plurality of image sensors. The prism 12a is an optical element that separates incident light into light components in two or more types of wavelength bands. The plurality of image sensors are imaging elements that receive the light components in two or more types of wavelength bands separated by the prism 12a, respectively. More specifically, the prism 12a separates incident light into light in the red and infrared wavelength bands, light in the green wavelength band, and light in the blue wavelength band. The image sensors 12b, 12c, and 12d that are the plurality of image sensors receive the light in the red and infrared wavelength bands, the light in the green wavelength band, and the light in the blue wavelength band, which are separated by the prism 12a, respectively. When pixels that have undergone the binning processing are regarded as one pixel on a group basis, the pixels of at least one image sensor of the image sensors 12b, 12c, and 12d are arranged with a shift of a half pixel in at least one direction of the horizontal direction and/or the vertical direction with respect to the pixels of at least one other image sensor.

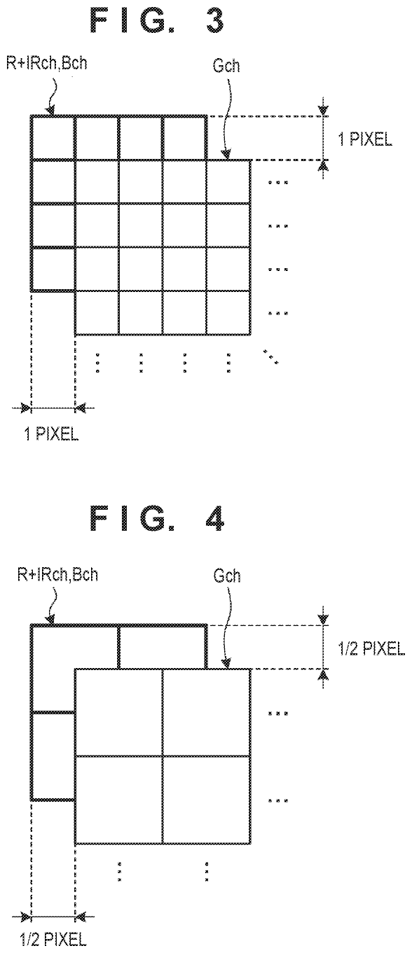

FIG. 3 is a view showing an example of the arrangement of the pixels of the image sensors 12b, 12c, and 12d of the imaging apparatus 10 according to this embodiment. As shown in FIG. 3, for example, the pixels of the image sensor 12c on the Gch side in the image sensors 12b, 12c, and 12d are arranged with a shift of one pixel in the horizontal direction and the vertical direction with respect to the pixels of the image sensor 12b on the R+IRch side and the image sensor 12d on the Bch side.

FIG. 4 is a view showing an example of the arrangement of the pixels of the image sensors 12b, 12c, and 12d when the imaging apparatus 10 according to this embodiment executes the ICG color mode. For example, in the ICG color mode, the control circuit 14a and the image sensor control circuit 12e cause the image sensors 12b, 12c, and 12d to execute binning processing when reading pixels. When the image sensors 12b, 12c, and 12d execute binning processing, for example, four adjacent pixels are added, and a group of two pixels in the horizontal direction x two pixels in the vertical direction is generated. In this case, when the pixels that have undergone the binning processing are regarded as one pixel on a group basis, as shown in FIG. 4, the pixels of the image sensor 12c on the Gch side are arranged with a shift of a half pixel in the horizontal direction and the vertical direction with respect to the pixels of the image sensor 12b on the R+IRch side and the image sensor 12d on the Bch side.

More specifically, in the normal mode, the image sensors 12b, 12c, and 12d read the pixels one by one in each row. In the ICG color mode, four adjacent pixels (two pixels in the horizontal direction x two pixels in the vertical direction) in two rows are added into one pixel, and the pixels after the addition are read. Hence, in the ICG color mode, the number of pixels to be read can be decreased by causing the image sensors 12b, 12c, and 12d to execute binning processing. Hence, in the ICG color mode, by executing binning processing, the image sensors 12b, 12c, and 12d can easily be driven at a high speed. In other words, the ICG color mode is a mode (high-speed read mode) in which binning processing is executed, and the normal mode is a mode in which binning processing is not executed.

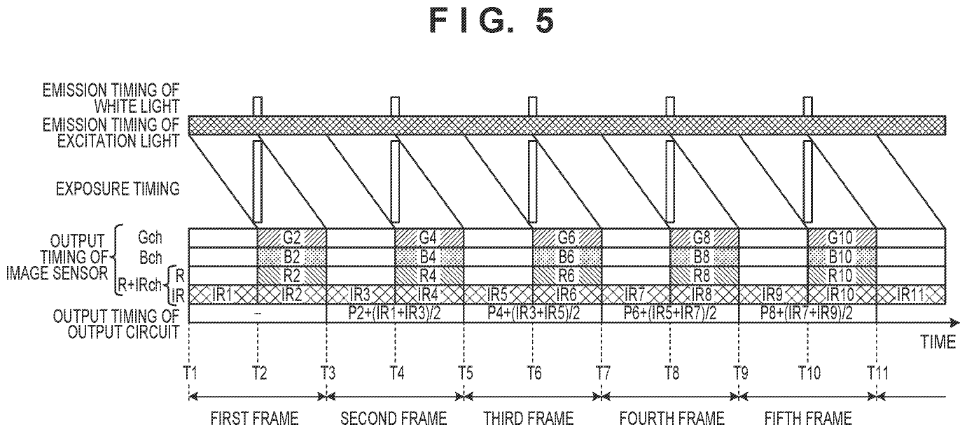

FIG. 5 is a view showing an imaging operation in the ICG color mode as an example of the imaging operation of the imaging apparatus 10 according to this embodiment. FIG. 5 shows an example of the relationship between the emission timings of white light and excitation light emitted from the light source apparatus 30, the exposure timings of the rows of the plurality of pixels provided in the image sensors 12b, 12c, and 12d of the imaging apparatus 10, the output timings of video signals output from the image sensors 12b, 12c, and 12d, and the output timings of a video signal output from the output circuit 14e. In FIG. 5, the abscissa represents time. In the ICG color mode, the frame rate (output rate) of a video signal (image) output from the imaging apparatus 10 to the display 101 is 60 [fps], as in the normal mode, and the read period is 1/120 [s]. That is, the period of outputting a video signal of one frame from the imaging apparatus 10 to the display 101 is 1/60 [s], and the read period is 1/120 [s].

In the ICG color mode, in each frame, time-divisional control is performed in which an R signal “IR” is acquired during the first read period of 1/120 [s], and an RGB signal is acquired during the second read period of 1/120 [s]. The imaging operation in the ICG color mode will be described below in detail.

First, at the start of imaging, the control circuit 14a outputs a control signal to the timing signal generation circuit 14f to cause it to output a first light source control signal that causes the IR laser 30d to continuously emit excitation light. The timing signal generation circuit 14f outputs the first light source control signal to the driving circuit 30c based on the control signal, and the driving circuit 30c drives the IR laser 30d based on the first light source control signal, thereby causing the IR laser 30d to continuously emit excitation light.

Also, in each frame, only during a blanking period that is a period when the first read period switches to the second read period, the control circuit 14a outputs a control signal to the timing signal generation circuit 14f to cause it to output a second light source control signal that causes the white LED 30b to emit white light. The timing signal generation circuit 14f outputs the second light source control signal to the driving circuit 30a based on the control signal, and the driving circuit 30a drives the white LED 30b based on the second light source control signal, thereby causing the white LED 30b to emit white light.

For example, in the first frame, during the first read period of 1/120 [s] from time T1 to time T2, a video signal (an R signal “IR1” to be described later) is output from the image sensor 12b. More specifically, the control circuit 14a outputs a control signal to the image sensor control circuit 12e to cause the image sensor 12b to output a video signal during the first read period of 1/120 [s]. The image sensor control circuit 12e drives and controls the image sensor 12b based on the control signal. As a result, during the read period of 1/120 [s] from time T2 to time T2, the image sensor 12b receives light in the infrared wavelength band, which has exited from the prism 12a, and outputs video signals from all rows as the R signal “IR1”. The storage control circuit 14b temporarily stores, in the storage circuit 14g, the video signal (R signal “IR1”) output from each row of the image sensor 12b.

Additionally, in the first frame, during the first read period of 1/120 [s] from time T1 to time T2, exposure is sequentially started on each row from the first row to the final row of the plurality of pixels of each of the image sensors 12b, 12c, and 12d. Here, a time difference corresponding to the read period is present between the exposure start and the exposure end (output start). For example, in the first row, exposure is performed during the period from the time T1 to the time T2, and output is performed at the time T2. In the second row, exposure is performed during the period from the time T2 to time T3, and output is performed at the time T3. More specifically, the control circuit 14a outputs a control signal to the image sensor control circuit 12e to cause the image sensors 12b, 12c, and 12d to execute binning processing and output video signals during the second read period of 1/120 [s]. The image sensor control circuit 12e drives and controls the image sensors 12b, 12c, and 12d based on the control signal. When the image sensors 12b, 12c, and 12d execute binning processing, four adjacent pixels are added, and a group of two pixels in the horizontal direction x two pixels in the vertical direction is generated. As a result, the image sensor 12b receives light in the red and infrared wavelength bands, which has exited from the prism 12a, and outputs video signals from all groups as an R signal “R2+IR2” during the second read period of 1/120 [s] from the time T2 to the time T3. The image sensor 12c receives light in the green wavelength band, which has exited from the prism 12a, and outputs video signals from all groups as a G signal “G2”. The image sensor 12d receives light in the blue wavelength band, which has exited from the prism 12a, and outputs video signals from all groups as a B signal “B2”. The storage control circuit 14b temporarily stores, in the storage circuit 14g, an RGB signal “P2” as the video signals output from the image sensors 12b, 12c, and 12d. The RGB signal “P2” represents the composite signal of the R signal “R2+IR2”, the G signal “G2”, and the B signal “B2”. That is, the RGB signal “P2” includes the R signal “R2+IR2” output from the image sensor 12b that has received the white light and the fluorescence based on the excitation light, the G signal “G2” output from the image sensor 12c that has received the white light, and the B signal “B2” output from the image sensor 12d that has received the white light. In other words, the RGB signal “P2” shown in FIG. 5 is a signal “W2+IR2” including the signal “W2=R2+G2+B2” based on the white light and the signal “IR2” based on the fluorescence.

In this way, the control circuit 14a drives and controls the image sensors 12b, 12c, and 12d in synchronism with the period when light is emitted from at least one light source (white LED 30b) of the plurality of light sources (the white LED 30b and the IR laser 30d). In addition, the number of pixels to be read can be decreased by causing the image sensors 12b, 12c, and 12d to execute binning processing. Note that the R signal “IR1” output during the first read period of 1/120 [s] from the time T1 to the time T2 has large scattering and is fluorescence, the resolution of an image represented by the R signal “IR1” is low, and a resolution is not needed as compared to an image represented by a signal based on white light. On the other hand, during the second read period of 1/120 [s] from the time T2 to the time T3, since binning processing is executed, the resolution of the image represented by the RGB signal “P2” lowers. For this reason, processing to be described later is executed for the pixels that have undergone the binning processing to obtain the same number of output pixels as in a case where binning processing is not executed. That is, by the processing to be described later, the number of output pixels of an image represented by the RGB signal “P2” becomes equal to the number of output pixels in a case where binning processing is not executed.

Similarly, in the second frame, the image sensor 12b outputs a video signal as an R signal “IR3” during the first read period of 1/120 [s] from the time T3 to time T4, and the storage control circuit 14b temporarily stores, in the storage circuit 14g, the video signal (R signal “IR3”) output from the image sensor 12b. Also, the image sensors 12b, 12c, and 12d output video signals as an R signal “R4+IR4”, a G signal “G4”, and a B signal “B4”, respectively, during the second read period of 1/120 [s] from the time T4 to time T5. The storage control circuit 14b temporarily stores, in the storage circuit 14g, an RGB signal “P4” as the video signals output from the image sensors 12b, 12c, and 12d. The RGB signal “P4” represents the composite signal of the R signal “R4+IR4”, the G signal “G4”, and the B signal “B4”.

Here, in the second frame, for example, the video signal of the first frame stored in the storage circuit 14g is output from the output circuit 14e to the display 101 via the image processing circuit 14c and the image composition circuit 14d. More specifically, the image processing circuit 14c generates a first display image based on the RGB signal “P2”. Next, the image composition circuit 14d composites, for example, the R signal “IR1” and the R signal “IR3”, thereby generating a composite image “(IR1+IR3)/2”. Next, the image composition circuit 14d extracts, as a target, a portion with a brightness equal to or larger than a threshold from the generated composite image, and generates a fluorescent image that is a marker formed by adding a fluorescent color to the extracted portion. The fluorescent color is a color assigned to represent fluorescence when the marker (fluorescent image) is generated, and shows, for example, green of high saturation. The image composition circuit 14d superimposes the generated fluorescent image on the first display image generated by the image processing circuit 14c, thereby generating a second display image. The second display image generated by the image composition circuit 14d is output from the output circuit 14e to the display 101 during the period of 1/60 [s]. From the third frame as well, processing similar to the above-described processing is performed.

Here, before the first display image is generated from the RGB signal “P2”, the image processing circuit 14c performs the following processing for the pixels that have undergone the binning processing to obtain the same number of output pixels as in a case where binning processing is not executed.

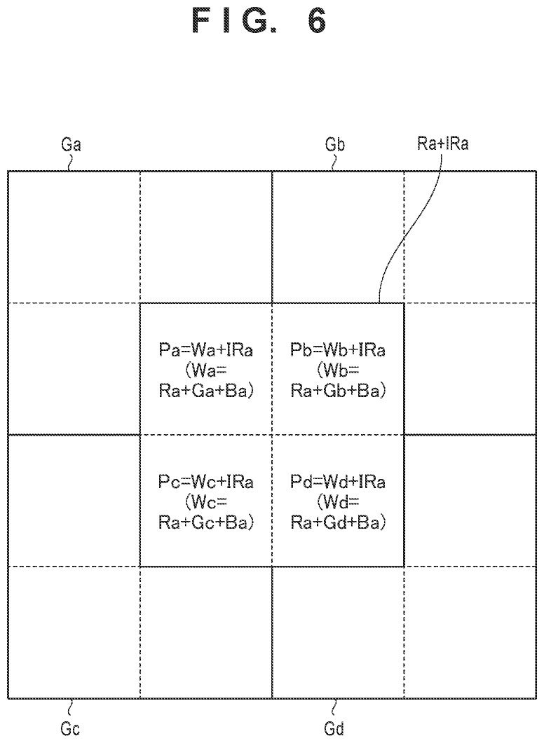

In the ICG color mode, since the pixels that have undergone the binning processing (in this embodiment, 4-pixel addition) are regarded as one pixel on a group basis, pixel values corresponding to four pixels are acquired from the pixels that have undergone the 4-pixel addition using the pixels of the image sensor 12c and the pixels of the image sensors 12b and 12d arranged with a shift of a half pixel. For example, as shown in FIG. 6, in a case where binning processing (4-pixel addition) is executed, pixels Ga, Gb, Gc, and Gd of the image sensor 12c on the Gch side are arranged with a shift of a half pixel with respect to a pixel Ra+IRa of the image sensor 12b on the R+IRch side and a pixel Ba of the image sensor 12d on the Bch side.

The image processing circuit 14c acquires the pixel value of a RGB signal “Pa” based on the pixel value of the pixel Ra+IRa, the pixel value of the pixel Ga, and the pixel value of the pixel Ba to obtain the same number of output pixels as in a case where binning processing is not executed (for example, to obtain the same number of output pixels as in the example shown in FIG. 3). The RGB signal “Pa” is a signal “Wa+IRa” including a signal “Wa=Ra+Ga+Ba” based on white light and a signal “IRa” based on fluorescence. Similarly, the image processing circuit 14c acquires the pixel value of a RGB signal “Pb” based on the pixel value of the pixel Ra+IRa, the pixel value of the pixel Gb, and the pixel value of the pixel Ba. The RGB signal “Pb” is a signal “Wb+IRa” including a signal “Wb=Ra+Gb+Ba” based on white light and the signal “IRa” based on fluorescence.

In addition, the image processing circuit 14c acquires the pixel value of a RGB signal “Pc” based on the pixel value of the pixel Ra+IRa, the pixel value of a pixel Gc, and the pixel value of the pixel Ba to obtain the same number of output pixels as in a case where binning processing is not executed. The RGB signal “Pc” is a signal “Wc+IRa” including a signal “Wc=Ra+Gc+Ba” based on white light and the signal “IRa” based on fluorescence. Similarly, the image processing circuit 14c acquires the pixel value of a RGB signal “Pd” based on the pixel value of the pixel Ra+IRa, the pixel value of a pixel Gd, and the pixel value of the pixel Ba. The RGB signal “Pd” is a signal “Wd+IRa” including a signal “Wd=Ra+Gd+Ba” based on white light and the signal “IRa” based on fluorescence.

In this way, the image processing circuit 14c acquires pixel values corresponding to four pixels from the pixels that have undergone the 4-pixel addition using the pixels of the image sensor 12c and the pixels of the image sensors 12b and 12d arranged with a shift of a half pixel, thereby obtaining the same number of output pixels as in a case where binning processing is not executed.

After the above-described processing, the image processing circuit 14c generates the above-described first display image. The image composition circuit 14d generates the above-described fluorescent image, and superimposes the fluorescent image on the first display image, thereby generating a second display image. At this time, the second display image is output from the output circuit 14e to the display 101 during the period of 1/60 [s].

As described above, in the imaging apparatus 10 according to this embodiment, in the ICG color mode, when pixels that have undergone binning processing are regarded as one pixel on a group basis, the pixels of the image sensor 12c on the Gch side are arranged with a shift of a half pixel in the horizontal direction and the vertical direction with respect to the pixels of the image sensor 12b on the R+IRch side and the image sensor 12d on the Bch side. That is, in the ICG color mode, pixel shift processing is performed. Hence, in the ICG color mode, lowering of the resolution caused by execution of binning processing is suppressed to some extent by the pixel shift. Accordingly, in the ICG color mode, the imaging apparatus 10 according to this embodiment can easily perform high-speed driving by binning processing and also suppress lowering of the resolution by pixel shift processing. Hence, in this embodiment, it is possible to ensure image quality sufficient for the user to observe.

Also, in the ICG color mode, instead of reading pixels as many as in the normal mode by the above-described time-divisional control, the imaging apparatus 10 according to this embodiment need only read a half the pixels in the normal mode by the above-described time-divisional control. It is therefore possible to suppress the number of signal processing operations. In this embodiment, the diameter per cable such as the camera cable 13 or the number of cables can be suppressed, and an increase in the diameter of the entire cable can be suppressed.

As described above, in this embodiment, after execution of binning processing, the pixels of the image sensor 12c on the Gch side are arranged with a shift of a half pixel in the horizontal direction and the vertical direction with respect to the pixels of the image sensor 12b on the R+IRch side and the image sensor 12d on the Bch side (see FIG. 4). To implement this, a case in which the pixels of the image sensor 12c on the Gch side are arranged with a shift of one pixel in the horizontal direction and the vertical direction with respect to the pixels of the image sensor 12b on the R+IRch side and the image sensor 12d on the Bch side before execution of binning processing has been described (see FIG. 3). In this case, as described above, in the ICG color mode, the image sensors 12b, 12c, and 12d add four adjacent pixels (two pixels in the horizontal direction x two pixels in the vertical direction) in two rows into one pixel, and read the pixels after the addition. However, the arrangement of the pixels before execution of binning processing is not limited to this.

For example, before execution of binning processing, the pixels of the image sensor 12c on the Gch side may be arranged with a shift of one pixel pitch or more with respect to the pixels of the image sensor 12b on the R+IRch side and the image sensor 12d on the Bch side. More specifically, before execution of binning processing, the pixels of the image sensor 12c on the Gch side are arranged with a shift of 1.5 pixels with respect to the pixels of the image sensor 12b on the R+IRch side and the image sensor 12d on the Bch side. In this case, the image sensors 12b, 12c, and 12d add nine adjacent pixels (three pixels in the horizontal direction x three pixels in the vertical direction) in three rows into one pixel, and read the pixels after the addition. In this case as well, when pixels that have undergone binning processing are regarded as one pixel on a group basis, the pixels of the image sensor 12c on the Gch side are arranged with a shift of a half pixel in the horizontal direction and the vertical direction with respect to the pixels of the image sensor 12b on the R+IRch side and the image sensor 12d on the Bch side.

Also, in this embodiment, the pixels are shifted in the horizontal direction and the vertical direction. However, the present invention is not limited to this. For example, the direction of shifting the pixels may be only the horizontal direction or may be only the vertical direction.

According to at least one embodiment described above, it is possible to ensure image quality sufficient for the user to observe.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-151778, filed Aug. 22, 2019, which is hereby incorporated by reference herein in its entirety.

What is claimed is:

1. An imaging apparatus comprising:
an optical element configured to separate incident light into light components in at least two types of wavelength bands; and
a plurality of imaging elements configured to receive the light components in the at least two types of wavelength bands separated by the optical element, respectively,
wherein when pixels that have undergone binning processing are regarded as one pixel on a group basis, pixels of at least one imaging element of the plurality of imaging elements are arranged with a shift of a half pixel in at least one direction of a horizontal direction and/or a vertical direction with respect to pixels of at least one other imaging element.

2. The apparatus according to claim 1, further comprising a switching control unit configured to switch between a first mode in which the binning processing is executed and a second mode in which the binning processing is not executed.

3. The apparatus according to claim 2, further comprising a processing unit configured to, in a case where the binning processing is executed, perform processing for the pixels that have undergone the binning processing to obtain the same number of output pixels as in a case where the binning processing is not executed.

4. The apparatus according to claim 2, further comprising a driving control unit configured to, in a case where the binning processing is executed, drive and control the plurality of imaging elements at a speed higher than in a case where the binning processing is not executed.

5. The apparatus according to claim 4, wherein in a case where the binning processing is executed, an output rate of outputting a video signal output from each of the plurality of imaging elements to a display unit is the same as in the case where the binning processing is not executed.

6. The apparatus according to claim 4, wherein in a case where the binning processing is executed, the driving control unit drives and controls the plurality of imaging elements in synchronism with a period when light is emitted from at least one light source.
